(19) United States
(12) Patent Application Publication
(10) Pub. No.: US 2019/0233869 A1
(43) Pub. Date: Aug. 1, 2019

(54) ENZYMATIC BIOSENSORS, HYDROGEL COMPOSITIONS THEREFOR, AND METHODS FOR THEIR PRODUCTION

(71) Applicants: HITACHI CHEMICAL COMPANY AMERICA, LTD., San Jose, CA (US); HITACHI CHEMICAL COMPANY, LTD., Tokyo (JP)

(72) Inventors: ANANDO DEVADOSS, IRVINE, CA (US); CUIHUA XUE, IRVINE, CA (US)

(21) Appl. No.: 16/328,173

(22) PCT Filed: Aug. 25, 2017
(86) PCT No.: PCT/US17/48634

§ 371 Date: Feb. 25, 2019

Related U.S. Application Data
(60) Provisional application No. 62/379,980, filed on Aug. 26, 2016.

(51) Int. Cl. C12Q 1/00; G01N 27/327; G01N 27/30

(52) U.S. Cl. CPC C12Q 1/004; G01N 27/308; G01N 27/3277; G01N 27/3271

(57) ABSTRACT

A biosensor (1) is disclosed that may include at least one electrode surface (3); a reagent layer (5) disposed on top of the at least one electrode surface (3) and a reagent layer (5) formed thereon. The reagent layer (5) is formed according to the principles of the present invention, and may include a redox enzyme, a redox polymer, and a cross-linked gel. The reagent layer (5) is structured to act as a conductive matrix that traps the redox polymer and enzyme at the electrode surface.

### FIELD

The present invention relates to, in general, reagent materials used to prepare sensors, such as enzyme-based electrochemical biosensors, sensors formed thereby, and methods of their fabrication and use.

### BACKGROUND

Electrochemical biosensors are widely used to determine the concentrations of biochemical analytes such as glucose, lactate, uric acid, etc. in blood and urine. In addition, they are also being explored for integration into wearable devices for detection of analytes in non-invasive biological fluids such as sweat, saliva, tears, etc.

A typical electrochemical biosensor utilizes a reagent layer on top of a current collector, usually known as anelectrode in this field of invention. This reagent layer encompass an enzyme capable of oxidizing or reducing the analyte and a redox mediator that can facilitate electron transfer between the enzyme and the electrode. The reagent layer can be either a single layer or multiple layers. For further description of electrochemical-based sensors, see for example, U.S. Pat. No. 6,299,757, US 2006/0042944, and US 2015/0053564.

The abovementioned reagent layer can contain either leachable or non-leachable reagents. U.S. Pat. No. 6,299,757 describes both kinds of reagent layers and US 2006/0042944 describes trapping polymeric mediators and enzyme using a dialysis membrane formed from polymers. The leachable reagent layer is limited in application, for analysis of samples ex-vivo, for example in the case of blood droplet obtained by pricking the tip of finger using a lancet needle and transferred to the biosensor. The non-leachable reagent layer can also be used for implantable devices and wearable devices, as the reagents do not interact with the body.

There have been attempts to attain such reagent layers by using polymeric mediators. But the polymeric mediators are often easily degraded under the electrochemical conditions, utilize expensive materials such as osmium, or require multiple steps to prepare reagent layers to prevent the polymeric mediators from leaching. In another case, there are redox polymers that can be prepared using aromatic backbones (e.g., polythiophene) to render electrochemical stability and alkyl backbones (e.g., polyvinyl) which cannot establish good electrical communication with the electrode surface. Therefore, there is a need to obtain a reagent layer that is stable under electrochemical sensing conditions, that can be prepared in preferably a simple manner (e.g., a single drop-casting step), and is not prone to leaching during sensing.

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

While certain aspects of conventional technologies have been discussed to facilitate disclosure of the invention, Applicants in no way disclaim these technical aspects, and it is contemplated that the claimed invention may encompass or include one or more of the conventional technical aspects discussed herein.

### SUMMARY

The present invention may address one or more of the problems and deficiencies of the prior art discussed above. However, it is contemplated that the invention may prove useful in addressing other problems and deficiencies, or provides benefits and advantages, in a number of technical areas. Therefore the invention should not necessarily be construed as being limited to addressing any of the particular problems or deficiencies discussed herein.

The present invention has demonstrated the following benefits and advantages: analyte sensitivity; resistant to degradation under electrochemical conditions; resistant to leaching; and ease of manufacture.

Thus, according to one aspect, the present invention provides a biosensor comprising: at least one electrode surface; a reagent layer disposed on top of the at least one electrode surface, the reagent layer comprising: a redox enzyme, a redox polymer, and a gel. The reagent layer can be structured to act as a conductive matrix that traps the redox polymer and enzyme at the electrode surface.

According to a further aspect, the present invention provides a method of manufacturing a biosensor constructed as described herein, wherein the method comprises: depositing the reagent layer on the electrode surface in a single application step, and wherein the single application step can comprise drop casting.

### BRIEF DESCRIPTION OF THE DRAWINGS

FIG. 1 is a schematic cross-sectional illustration of a biosensor electrode formed according to the present invention.

FIG. 2 is a schematic illustration of the various components usable in a biosensor according to certain aspects of the present invention.

FIG. 3 is a schematic illustration of an exemplary biosensor construction utilizing the components depicted in FIG. 2 according to certain aspects of the present invention.

FIG. 4 is a schematic illustration of the various components usable in a biosensor according to further aspects of the present invention.

FIG. 5 is a schematic illustration of exemplary biosensor construction utilizing the components depicted in FIG. 4 according to certain aspects of the present invention.

FIG. 6 is plot of current and potential (voltage) for an electrode containing glucose dehydrogenase (“GDH”) and Fc-Thiophene-1, without carbon black, before (solid line) and after (broken line) glucose detection for 12 min at 0.4 V, vs a reference electrode (SCE), in pH 5.3, 0.1 M potassium phosphate, 26 mM sodium chloride, 10 mM glucose solution.

FIG. 7 is a plot of current and potential (voltage) for an electrode containing GDH-Fc-Thiophene-1-carbon black, electrode before (solid line) and after(broken line) glucose detection for 60 min at 0.4 V, vs a reference electrode (SCE), in pH 5.3, 0.1 M potassium phosphate, 26 mM sodium chloride, 10 mM glucose solution.

FIG. 8 is a plot of current versus time, for a glucose biosensor output at various concentrations of glucose according to additional aspects of the present invention.

FIG. 9 is a plot of current versus time, for lactate biosensor output at various concentrations of lactate according to additional aspects of the present invention.

FIG. 10 is a plot of current and potential (voltage) between the biosensor of FIG. 4 and a reference electrode, upon the application of voltage to a sample containing no lactate (broken line), and to a sample containing 25 mM lactate (solid line) according to further aspects of the present invention.

FIG. 11 is plot of current and potential (voltage) between a biosensor containing lactate oxidase, horseradish peroxidase, Fc-Thiophene-1, with and without carbon black, Trimethylolpropane tris[poly(propylene glycol), amine terminated] ether Mn 440, poly(ethylene glycol) diglycidyl ether Mn 500, and a reference electrode, in a 25 mM lactate solution, according to additional aspects of the present invention.

FIG. 12 is a plot of current versus time, for a lactate biosensor output at various concentrations of lactate, according to additional aspects of the present invention.

FIG. 13 depicts the current response of a lactate biosensor constructed according to further aspects of the present invention over time when exposed to a sample containing a concentration of 12 mM of lactate.

FIG. 14 depicts the current response of a lactate biosensor constructed according to additional aspects of the present invention when exposed to different concentrations of lactate at different temperatures.

### DETAILED DESCRIPTION

As used herein, the singular forms “a,” “an” and “the” are intended to include the plural forms as well, unless the context clearly indicates otherwise. Additionally, the use of “or” is intended to include “and/or”, unless the context clearly indicates otherwise.

As used herein, the term “redox enzyme” refers to an enzyme which catalyzes either oxidation or reduction of a substrate and during the process undergoes an electron transfer between the substrate and the co-factor of the enzyme

As used herein, the term “redox mediator” refers to a chemical moiety capable of undergoing oxidation or reduction through electron transfer with an electrode and with a redox enzyme.

As used herein, the term “redox polymer” refers to a polymer modified with a redox mediator.

As used herein, the term “hydrogel” refers to a polymeric network that is capable of swelling when exposed to water, thereby, allowing water to fill the empty space trapped between the network.

As used herein, the term “ionomer” is a polymer that comprises of predominantly electrically neutral repeating units and a fraction (e.g., 15% or less) of electrically charged repeating units. For example, the ionomer may be a sulfonated tetrafluoroethylene based fluoropolymer-copolymer (e.g., Nafion®) or a copolymer of ethylene and methacrylic acid (e.g., Surlyn®).

As used herein, the term “polyelectrolyte” is a polymer that comprises predominantly electrically charged repeating units (e.g., 30-100%).

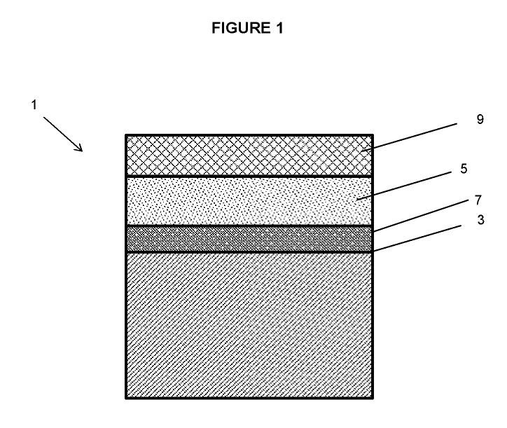

As illustrated in FIG. 1, a biosensor 1 may include at least one electrode surface 3; a reagent layer 5 disposed on top of the at least one electrode surface 3 and a reagent layer 5 formed thereon. The electrode surface 3 can be formed from any suitable material, such as carbon, or an allotrope thereof. The reagent layer 5 is formed according to the principles of the present invention, and may include a redox enzyme, a redox polymer, and a gel. Gels formed by physical bonds (physical gels) and/or gels formed by chemical bonds (chemical gels) are encompassed by the present invention. The reagent layer 5 is structured to act as a conductive matrix that traps the redox polymer and enzyme at the electrode surface. The biosensor one may further include an optional current capture layer 7. When the current capture layer 7 is absent, the reagent layer 5 may be formed directly upon the surface of the electrode 3. The biosensor 1, may further comprise an optional second layer 9 on top of the reagent layer 5. The second layer 9 may have any suitable composition, such as a hydrogel. One non-limiting example of a suitable composition for the second layer 9 is an enzyme-containing hydrogel layer comprising, for example, 4-styrene sulfonic acid-co-maleic acid and polyethylene glycol diglycidyl ether. When the second layer 9 is absent, the reagent layer 5 may form the uppermost layer of the biosensor 1.

According to a further aspect, the present invention provides a method of manufacturing a biosensor constructed as described herein, wherein the method comprises: depositing the reagent layer on the electrode surface in a single application step, and wherein the single application step can comprise drop casting.

In the present invention, compositions for forming a electrically conductive reagent layers for electrochemical biosensor containing polymeric redox-mediator, carbon nanomaterial and enzyme or enzymes entrapped using cross-linkable molecules in one-step is presented. In addition, the formed reagent layers show enhanced stability of the redox mediator, and enhanced electrical communication between the redox mediator and the electrode during the electrochemical biosensing process.

In one embodiment of the present invention, at least one electrode surface is present, the said electrode surface is coated with a film, thus forming a reagent layer, using a simple technique. The reagent layer includes at least one redox enzyme, carbon nanomaterial as a conductive matrix dispersed using a dispersing aid, such as an ionomer (e.g., Nafion® or Surlyn®), and a redox polymer either water-soluble or non-water soluble and cross-linked molecules. The cross-linked molecules trap the polymeric redox mediators in a gel-like film and prevent them from leaching. In addition, the carbon nanomaterial forms a porous conductive matrix that can provide facile electrical communication between the redox mediators and electrode surface during the electrochemical biosensing.

Furthermore, the molecules used to form gel layer(s) are also chosen carefully so as not to swell in presence of aqueous fluids to an extent where the expansion in the gel-layer or reagent layer causes loss of electrical communication between the carbon nanomaterial and electrode surface.

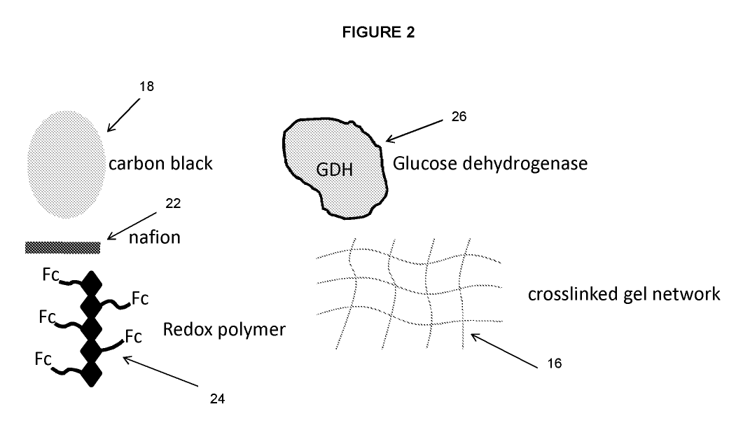

Certain features, functionalities, benefits and advantages associated with the present invention are further illustrated in FIGS. 2-14.

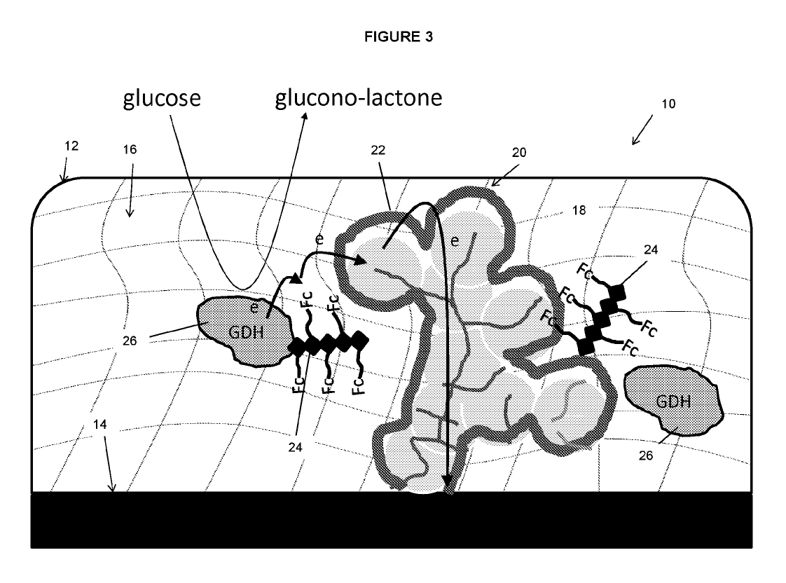

FIGS. 2-3 illustrate an exemplary, nonbinding, sensor construction designed for sensing glucose. The glucose biosensor 10 generally may include a reagent layer 12 disposed on a surface of an electrode 14, such as a carbon electrode. The reagent layer 12 is formed by a cross-linked gel network 16 containing a number of additional constituents.

The cross-linked gel network 16 can be formed of any suitable material. According to one embodiment, the cross-linked gel network 16 is formed by a hydrogel. Suitable examples of gel network 16 materials include the following compounds:

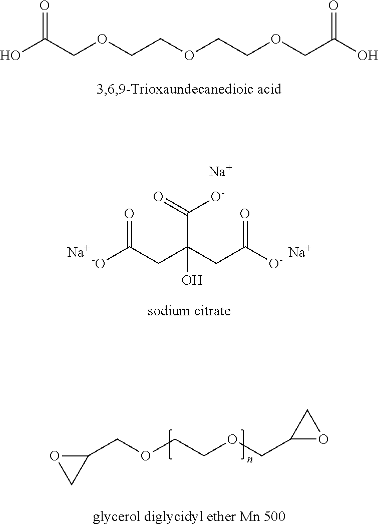

The above compounds can be utilized independently or in combination with each other, or in combination with other materials.

The reagent layer 12/gel network 16 optionally includes one or more carbon nanomaterials 18 therein. When present, the carbon nanomaterials 18 can be in any suitable form. Suitable nonlimiting examples include: carbon black (1-300 nm in diameter); carbon nanotubes (single or multiwalled; 0.3-100 nm in diameter); carbon nanofiber (1-200 nm in diameter); graphene (1-500 nm); and graphite nanopowder. When present, the carbon nanomaterials can form an aggregate 20 within the gel network 16, as illustrated in FIG. 3.

As further illustrated in FIG. 3, ionomer 22 can also be present in the gel network 16. Optionally, the ionomer 22 may surround the aggregate of carbon nanomaterials 20. Any suitable ionomer 22 can be utilized. Suitable nonlimiting examples include a sulfonated tetrafluoroethylene based fluoropolymer-copolymer, such as Nafion®, or a copolymer of ethylene and methacrylic acid, such as Surlyn®.

The reagent layer 12/gel network 16 additionally includes a redox polymer 24. Any suitable redox polymer 24 can be utilized. According to certain embodiments, the redox polymer 24 comprises a ferrocene-containing polymer. According to further embodiments, the redox polymer 24 comprises a tetrathiafulvalene (TTF)-containing polymer. The redox polymer may optionally be characterized as comprising a backbone comprising a conjugated polymer, a first side chain attached to the backbone, the first side chain comprising a ferrocene group, a tetrathiafulvalene group or derivatives thereof, a second side chain attached to the backbone, the second side chain comprising an organic acid or a salt of an organic acid, and at least one of the first and second side chains comprising at least one of a carbon atom, a nitrogen atom, an oxygen atom, and a sulfur atom. The conjugated polymer may optionally comprise at least one of a polythiophene, a polyaniline, a polyacetylene, a poly(p-phenylene), a polypyrrole and derivatives thereof. The first chain may optionally comprise 5 to 40 atoms between the ferrocene group, the tetrathiafulvalene group, or the derivatives thereof, and the conjugated polymer. At least one of the first and second side chains may further optionally comprise an ethylene oxide group. The second side chain can optionally comprise a carboxylic acid group, a carboxylate group, a sulfonic acid group or a sulfonate group. According to one optional embodiment, the redox polymer is water soluble.

Further, according to certain additional nonlimiting embodiments, the redox polymer 24 can comprise any of the following compounds (A)-(G).

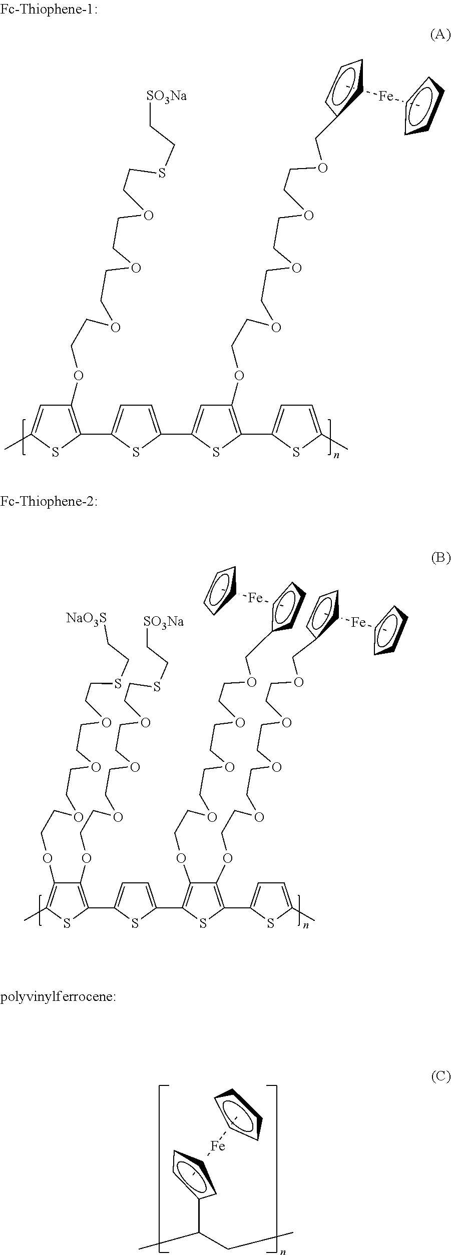

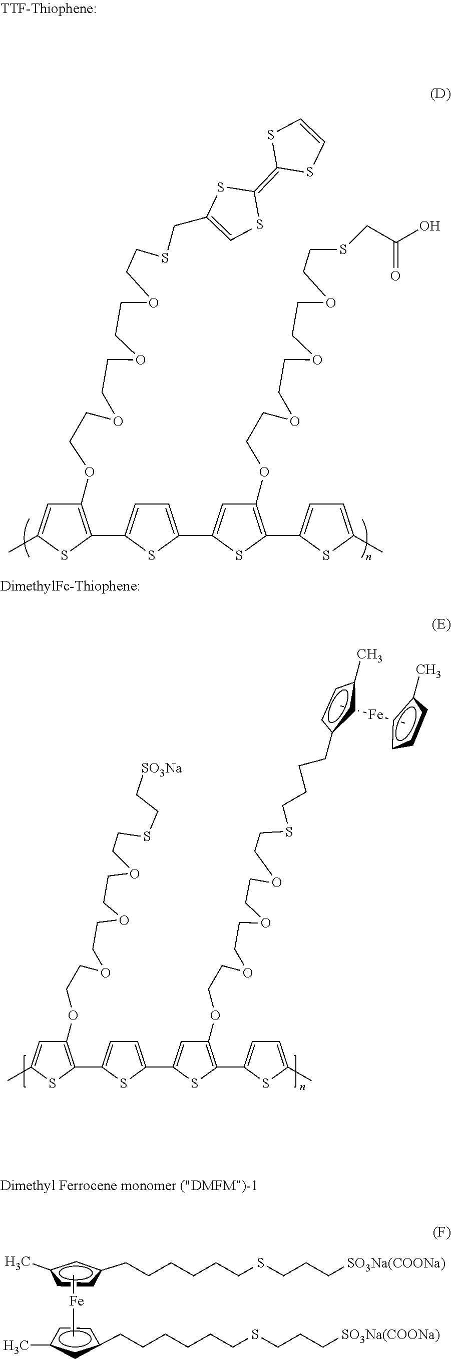

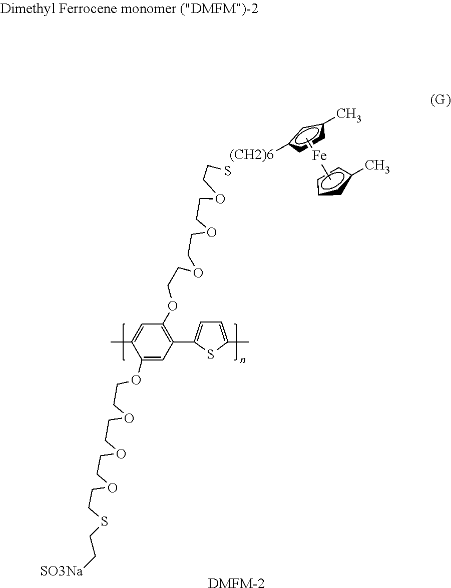

Additional optional redox polymers that may be utilized consistent with the principles of the present invention are described in copending Application Ser. No. 62/379,509, the entire contents of which are incorporated herein by reference.

Finally, the reagent layer 12/gel network 16 includes a redox enzyme. Suitable redox enzymes include at least one of: a dehydrogenase, a reductase, an oxidase, an oxygenase, a peroxidase, a catalase and a transhydrogenase. When in the form of the glucose biosensor 10, the redox enzyme, can comprise, for example, glucose dehydrogenase. Glucose dehydrogenase is, for example, an enzyme that catalyzes the following chemical reaction: D-glucose+acceptor⇄D-glucono-1,5-lactone+reduced acceptor. Thus, the two products of the reaction are D-glucono-1,5-lactone and reduced acceptor. Any suitable glucose dehydrogenase can be utilized. Alternatively, a glucose oxidase may be used instead of a glucose dehydrogenase.

As schematically illustrated in FIG. 3, the redox polymer 24 can function to trap the glucose dehydrogenase 26 within the gel network 16 of the reagent layer 12, thereby preventing and/or mitigating undesirable leaching.

As previously noted In addition to the reagent layer 12 containing enzymes, in some cases it is advantageous to add a non-enzymatic additional layer (e.g., a second layer 9; FIG. 1) to improve the performance of the biosensor. One example would be to add a second layer on top of the reagent layer 12 that has a net negative charge to limit the concentration of a negatively charged analyte (e.g., lactate) in the enzyme-containing hydrogel reagent layer. This can improve the enzyme stability and also to limit the concentration of interferents such as ascorbate and uric acid from reaching the electrode surface and giving false signals.

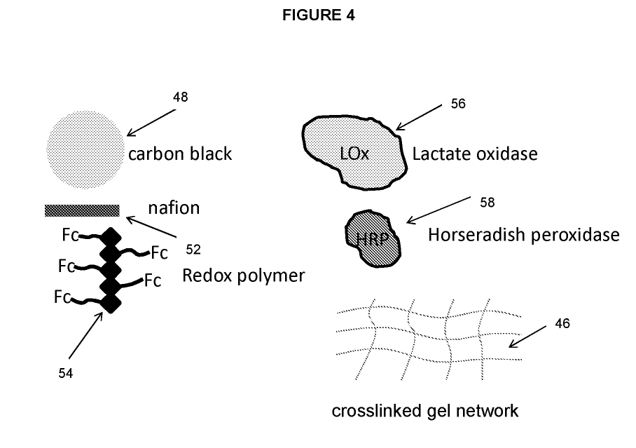

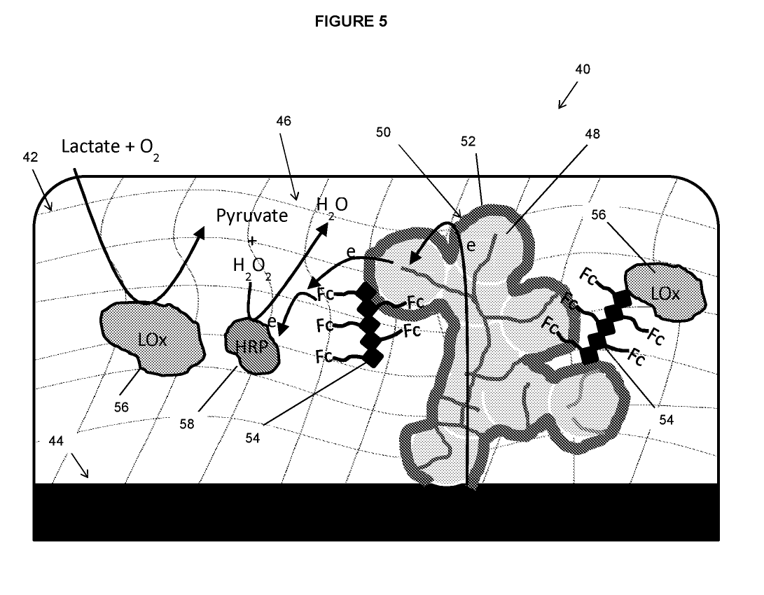

FIGS. 4-5 illustrate an exemplary, nonbinding, sensor construction designed for sensing lactate. The lactate biosensor 40 generally may include a reagent layer 42 disposed on a surface of an electrode 44, such as a carbon electrode. The reagent layer 42 is formed by a cross-linked gel network 46 containing a number of additional constituents.

The cross-linked gel network 46 can be formed of any suitable material. According to one embodiment, the cross-linked gel network 46 is formed by a hydrogel. Suitable examples of gel network 46 materials include the following compounds:

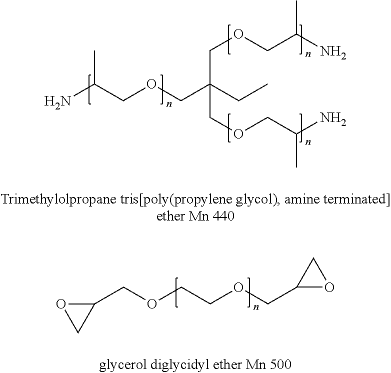

The above compounds can be utilized independently or in combination with each other, or with other materials. In addition, the gel compounds previously described herein for use in connection with the glucose biosensor 10 can also be utilized in formation of the lactate biosensor 40. Likewise, the glucose biosensor 10 can utilize the above-mentioned gel compounds in the formation of the gel network 16.

The reagent layer 42/gel network 46 optionally includes one or more carbon nanomaterials 48 therein. When present, the carbon nanomaterials 48 can be in any suitable form. Suitable nonlimiting examples include: carbon black (1-300 nm in diameter); carbon nanotubes (single or multiwalled; 0.3-100 nm in diameter); carbon nanofiber (1-200 nm in diameter); graphene (1-500 nm); and graphite nanopowder. When present, the carbon nanomaterials can form an aggregate 50 within the gel network 46, as illustrated in FIG. 4.

As further illustrated in FIG. 4, and ionomer 52 can also be present in the gel network 46. Optionally, the ionomer 52 may surround the aggregate of carbon nanomaterials 50. Any suitable ionomer 52 can be utilized. Suitable nonlimiting examples include a sulfonated tetrafluoroethylene based fluoropolymer-copolymer, such as Nafion®, or a copolymer of ethylene and methacrylic acid, such as Surlyn®.

The reagent layer 42/gel network 46 additionally includes a redox polymer 54. Any suitable redox polymer 54 can be utilized, one non-limiting example being a polyetheramine, such as Jeffamine® can be utilized. According to certain embodiments, the redox polymer 54 comprises a ferrocene-containing polymer or a tetrathiafulvalene (TTF)-containing polymer. The redox polymer may optionally be characterized as comprising a backbone comprising a conjugated polymer, a first side chain attached to the backbone, the first side chain comprising a ferrocene group, a tetrathiafulvalene group or derivatives thereof, a second side chain attached to the backbone, the second side chain comprising an organic acid or a salt of an organic acid, and at least one of the first and second side chains comprising at least one of a carbon atom, a nitrogen atom, an oxygen atom, and a sulfur atom. The conjugated polymer may optionally comprise at least one of a polythiophene, a polyaniline, a polyacetylene, a poly(p-phenylene), a polypyrrole and derivatives thereof. The first chain may optionally comprise 5 to 40 atoms between the ferrocene group, the tetrathiafulvalene group, or the derivatives thereof, and the conjugated polymer. At least one of the first and second side chains may further optionally comprise an ethylene oxide group. The second side chain can optionally comprise a carboxylic acid group, a carboxylate group, a sulfonic acid group or a sulfonate group. According to one optional embodiment, the redox polymer is water soluble.

Further, according to certain additional nonlimiting embodiments, the redox polymer can comprise any of the compounds (A)-(G), as defined above. Additional optional redox polymers that may be utilized consistent with the principles of the present invention are described in copending Application Ser. No. 62/379,509, the entire contents of which are incorporated herein by reference.

Finally, the reagent layer 42/gel network 46 of the lactate biosensor 40 includes at least one redox enzyme (56, 58). Suitable redox enzymes include at least one of: a dehydrogenase, a reductase, an oxidase, an oxygenase, a peroxidase, a catalase and a transhydrogenase. When in the form of the lactate biosensor 40, the redox enzyme, can comprise, for example, a lactate oxidase 56, and a horseradish peroxidase 58. Lactate oxidase 56 is an enzyme that catalyzes the chemical reaction: (S)-lactate+O₂⇄pyruvate+H₂O₂. Any suitable lactate oxidase can be utilized. Horseradish peroxidase reduces H₂O₂ by catalyzing the following reaction, H₂O₂+donor⇄H₂O+oxidized-donor. In this case the donor can be any redox mediator (e.g., ferrocene) in a reduced state. By using both lactate oxidase and hydrogen peroxidase in combination, lactate can be detected indirectly by detecting H₂O₂. Any suitable horseradish peroxidase, or conjugate thereof, can be utilized.

As schematically illustrated in FIG. 5, the redox polymer 54 can function to trap the lactate oxidase 56 within the gel network 46 of the reagent layer 42, thereby preventing and/or mitigating undesirable leaching.

In accordance with the above-mentioned teachings, a number of different redox polymers are evident. The following are illustrative, nonlimiting examples of suitable redox polymer formulations consistent with the principles of the present invention.

### Synthesis of Redox Polymer (A) (Fc-Thiophene-1)

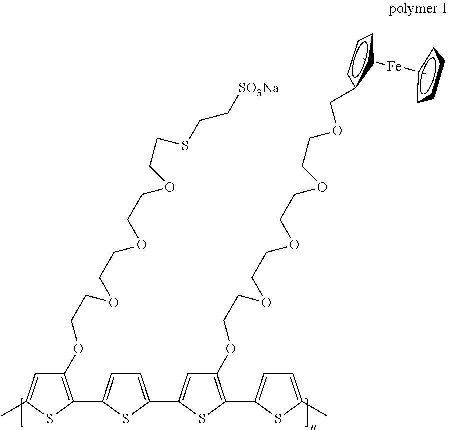

Scheme 1 below illustrates the synthesis of monomer 1: 2,5-dibromo-3-(2-(2-(2-(2-bromoethoxy)ethoxy)ethoxy)ethoxy)thiophene.

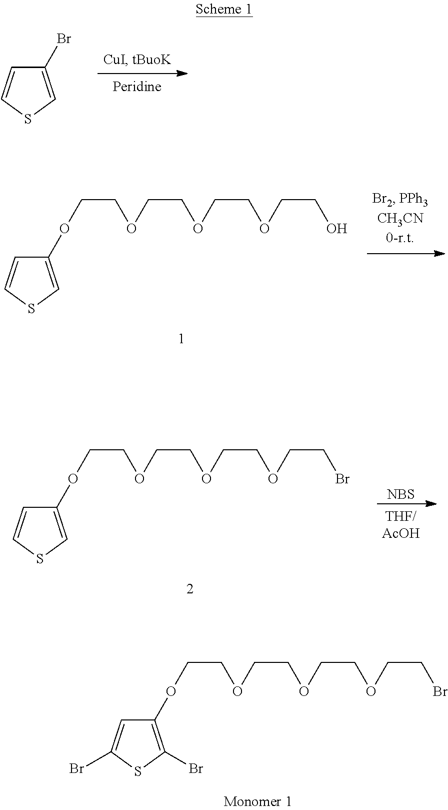

To a 500 mL three-necked round-bottomed flask, t-BuOK (26 g, 232 mmol), CuI (6.0 g, 31.6 mmol), pyridine (30 mL) and 2,2′-((oxybis(ethane-2,1-diyl))bis(oxy))diethanol (149 g, 767 mmol) were added. The mixture was stirred at room temperature for 30 minutes under a nitrogen atmosphere and then 3-bromothiophene (25.0 g, 153.4 mmol) was added. The mixture was then heated to 100° C. for about 24 hrs until disappearance of the 3-bromothiophene, as monitored by TLC. The reaction mixture was cooled to room temperature, poured into 10% HCl solution, extracted with ethyl acetate (“EtOAc”), washed with 10% NH₄Cl solution and/or NaCl solution, and dried over anhydrous MgSO₄. After removal of the solvent, the crude mixture was purified by chromatography to give compound 1 as an oil.

PPh₃ (9.5 g, 36.3 mmol) was suspended in 30 mL of CH₃CN under a nitrogen atmosphere at 0° C. and Br₂ (2.9 g, 18.12 mmol) was slowly added. Then, compound 1 (5 g, 18.12 mmol) in 10 mL CH₃CN was added dropwise and the mixture was stirred from 0° C. to room temperature for about 48 hrs. Any remaining solid was filtered and the filtrate was purified by chromatography to provide compound 2 as an oil.

Compound 2 (4.15 g, 12.24 mmol) was dissolved in a mixture of 8 mL THF and 8 mL AcOH. N-Bromosuccinimide (4.58 g, 25.73 mmol) was added and the mixture was stirred at room temperature for about 3 hrs. The reaction mixture was then poured into NaCl solution and extracted with EtOAc. Combined EtOAc was washed with NaCl solution and dried over anhydrous MgSO₄. After removal of the solvent, the crude mixture was purified by chromatography to give monomer 1.

Scheme 2 below illustrates the synthesis of monomer 2.

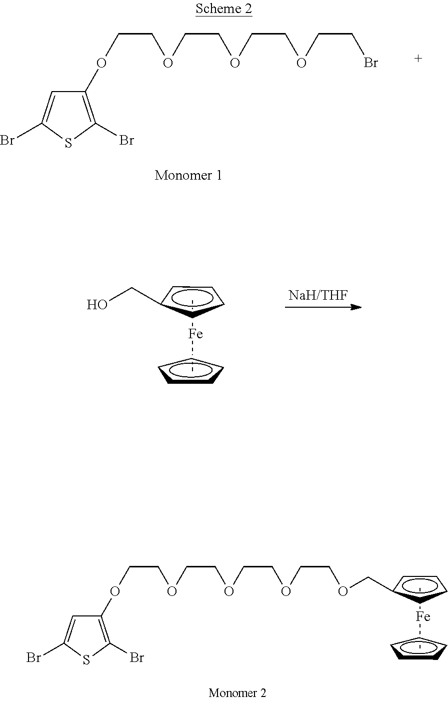

Ferrocenemethanol (4.8 g, 22.2 mmol) was dissolved in dry THF and NaH (0.8 g, 33.3 mmol) was added. The mixture was stirred at room temperature for about 20 minutes and then monomer 1 (10 g, 20.1 mmol) was added. The resulting mixture was stirred at room temperature for about 20 hrs until the disappearance of monomer 1, as monitored by TLC. The reaction mixture was then poured into NaCl solution and extracted with EtOAc. Combined EtOAc was washed with NaCl solution and dried over anhydrous MgSO₄. After removal of the solvent, the crude mixture was purified by chromatography to give monomer 2.

Scheme 3 below shows the co-polymerization of monomer 1, thiophene-2,5-diboronic acid and monomer 2 to produce polymer (A) precursor and polymer (A).

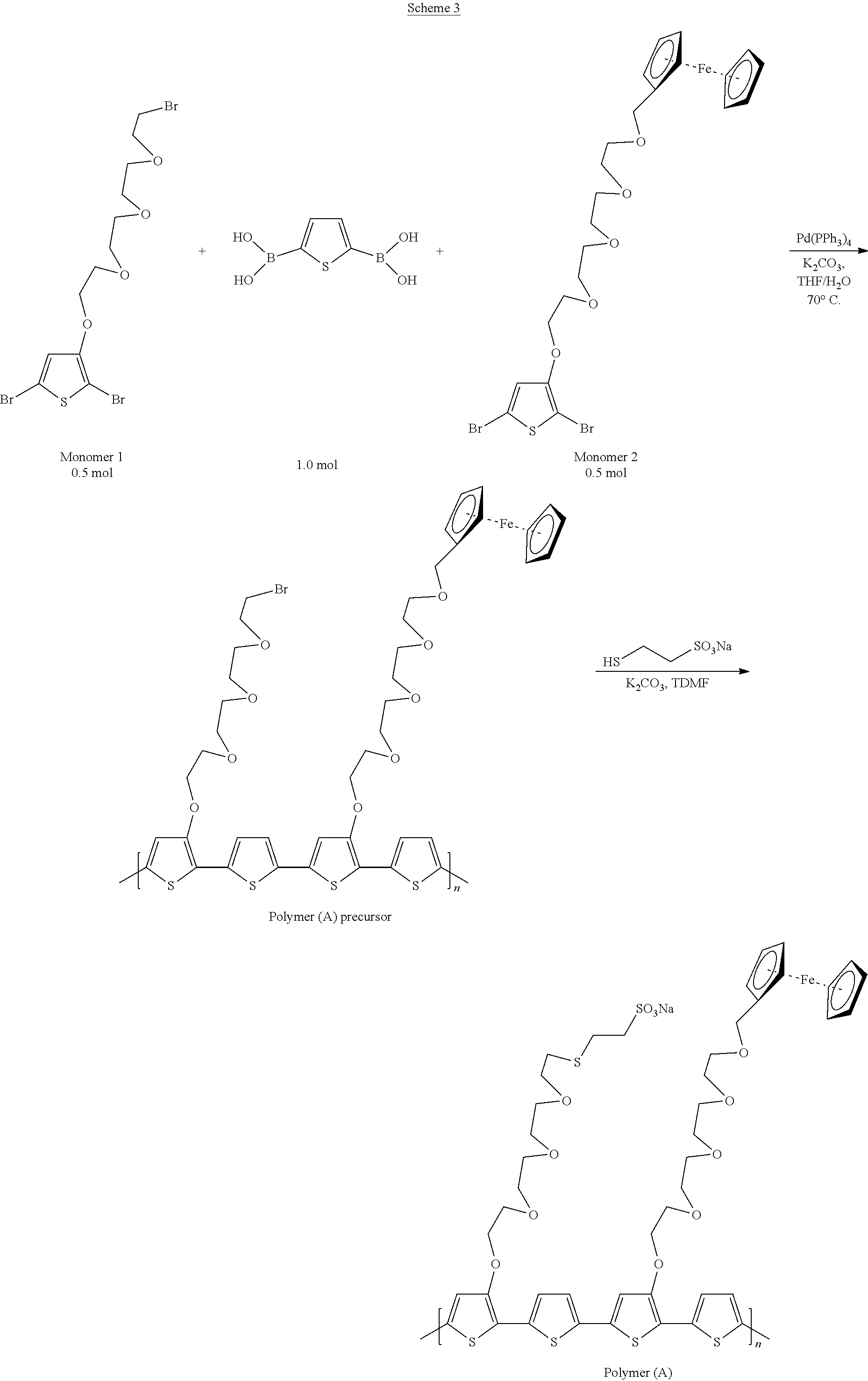

0.5 mol of monomer 1, 0.5 mol of monomer 2, 1.0 mol of 2,5-thiophene-diboronic acid, Pd (PPh₃)₄ (5% of monomer 1), and K₂CO₃ were placed in a two-necked flask under a nitrogen atmosphere. 20 ml of THF and 6 ml of water were added, and the reaction mixture was heated to 70° C. for about 20 h. The reaction was cooled to room temperature and poured into CH₃OH, which resulted in the formation of a precipitate. The collected precipitate was washed with CH₃OH several times and dried by vacuum to give polymer (A) precursor as a dark sticky oil. The polymer (A) precursor was then dissolved in anhydrous DMF, and 2 equivalents of K₂CO₃ and 2 equivalents of sodium 2-mercaptoethanesulfonate were added. The mixture was stirred at room temperature for about 16 hrs, and transferred into a dialysis tube (CO 12,000) for dialysis against water. After dialysis, the solution in the dialysis tube was filtered to remove insoluble impurities and then freeze-dried to give polymer (A).

### Synthesis of Redox Polymer (B) (Fc-Thiophene-2)

Scheme 4 below illustrates the synthesis of monomer 3:

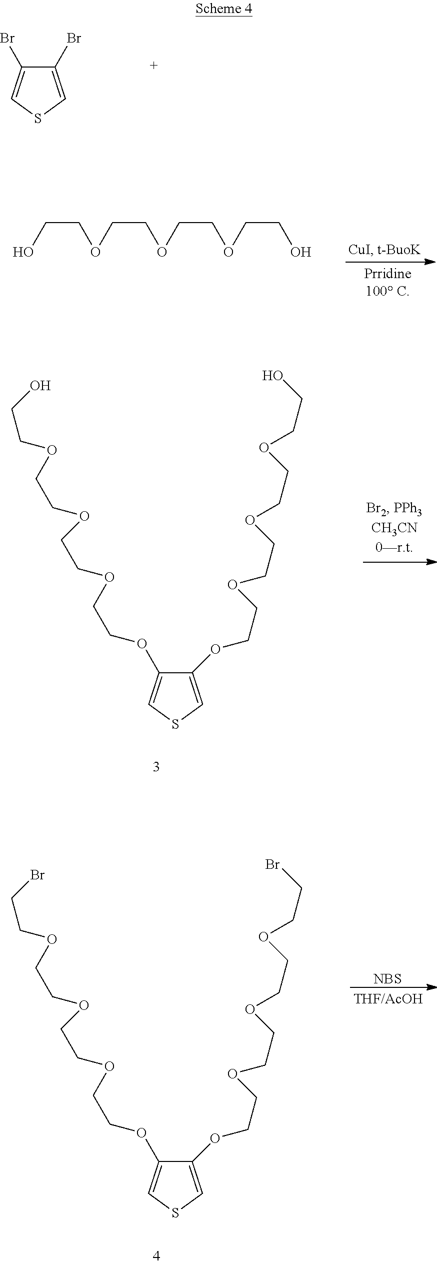 [SMILES: Brc1cscc1Br ; OCCOCCOCCOCCO ; OCCOCCOCCOCCOc1cscc1OCCOCCOCCOCCO ; BrCCOCCOCCOCCOc1cscc1OCCOCCOCCOCCBr ; "NBS"]

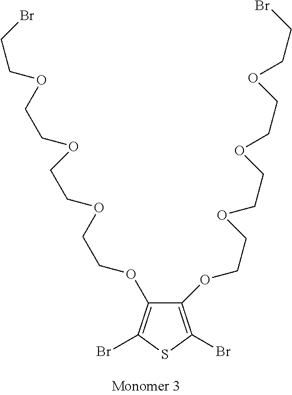 [SMILES: BrCCOCCOCCOCCOc1c(Br)sc(Br)c1OCCOCCOCCOCCBr]

To a 500 mL three-necked round-bottomed flask, t-BuOK (34 g, 0.3 mol), CuI (8.0 g, 40 mmol), pyridine (50 mL) and 2,2′-((oxybis(ethane-2,1-diyl))bis(oxy))diethanol (200 g, 1.03 mol) were added. The mixture was stirred at room temperature for about 30 minutes under a nitrogen atmosphere. Then, 3,4-dibromothiophene (25.0 g, 0.1 mmol) was added, and the mixture was heated to 100° C. for about 24 hrs until the disappearance of 3,4-dibromothiophene, as monitored by TLC. The reaction mixture was cooled to room temperature, poured into 10% HCl solution, and extracted with ethyl acetate (EtOAc). The combined EtOAc solution was washed with 10% saturated NH₄Cl solution and/or NaCl solution and dried over anhydrous MgSO₄. After removal of the solvent, the crude mixture was purified by chromatography to give compound 3 as an oil.

PPh₃ (18.8 g, 71.76 mmol) was suspended in 40 mL of CH₃CN under a nitrogen atmosphere at 0° C. and Br₂ (5.75 g, 35.94 mmol) was slowly added. After all of the Br₂ was added, compound 3 (8.4 g, 17.95 mmol) in 15 mL CH₃CN was added dropwise and the mixture was stirred from 0° C. to room temperature for about 48 hrs. After completion of the reaction, the solid in the mixture was filtered out and the filtrate was collected and purified by chromatography to provide compound 4 as an oil.

Compound 4 (7.6 g, 12.79 mmol) was dissolved in a mixture of 10 mL THF and 10 mL AcOH. N-Bromosuccinimide (4.78 g, 26.85 mmol) was added to the mixture, and the mixture was stirred at room temperature for about 4 hrs. The reaction mixture was then poured into NaCl solution and extracted with EtOAc. Combined EtOAc was washed with NaCl solution and dried over anhydrous MgSO₄. After removal of the solvent, the crude mixture was purified by chromatography to give monomer 3.

Scheme 5 below illustrates the synthesis of monomer 4.

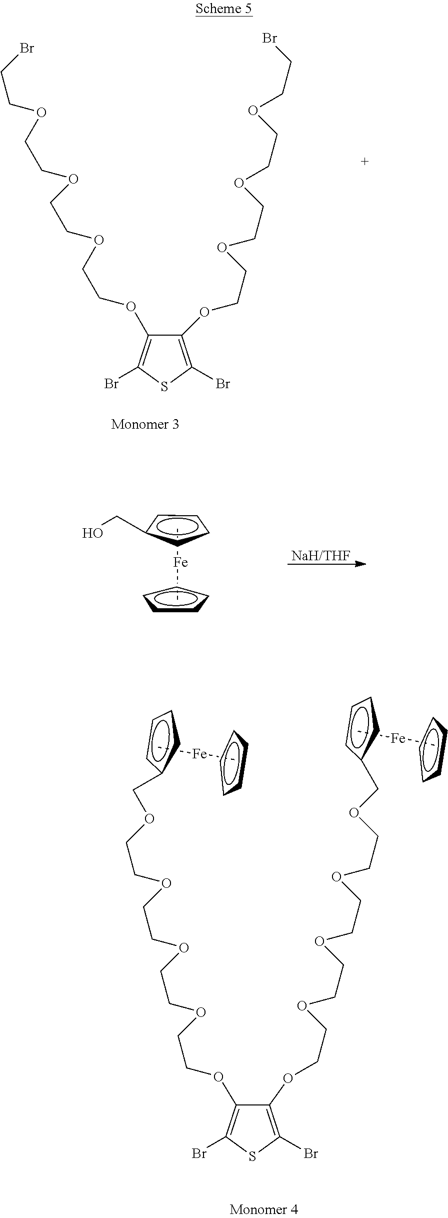

Ferrocenemethanol (2.3 g, 10.65 mmol) was dissolved in dry THF and NaH (0.25 g, 10.41 mmol) was added. The mixture was stirred at room temperature for about 20 minutes and then monomer 3 (3.0 g, 3.99 mmol) was added. The mixture was then stirred at room temperature for about 20 hrs until the disappearance of monomer 3, as monitored by TLC. The reaction mixture was then poured into NaCl solution and extracted with EtOAc. Combined EtOAc was washed with NaCl solution and dried over anhydrous MgSO₄. After removal of the solvent, the crude mixture was purified by chromatography to give monomer 4.

Scheme 6 below shows the co-polymerization of monomer 3, thiophene-2,5-diboronic acid and monomer 4 to produce polymer (B) precursor and polymer (B).

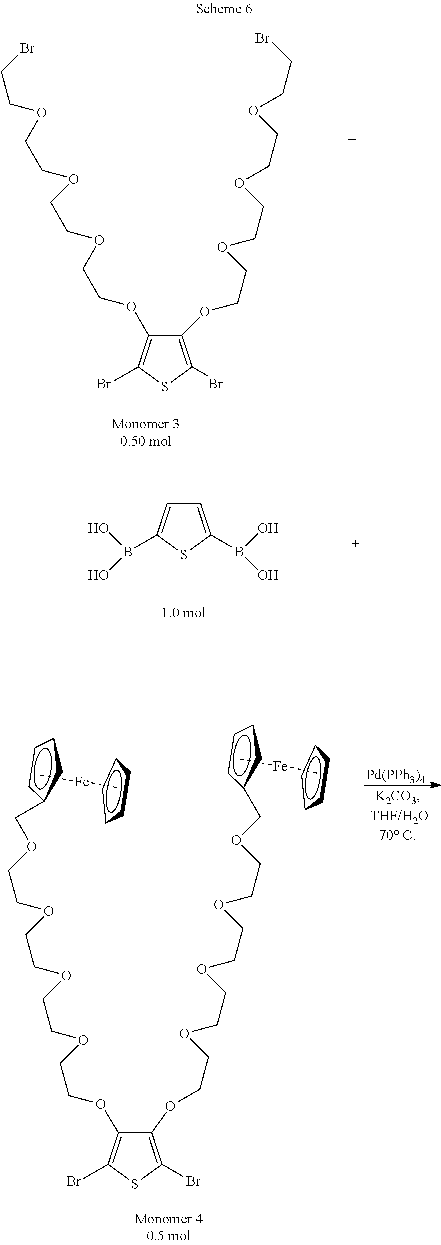

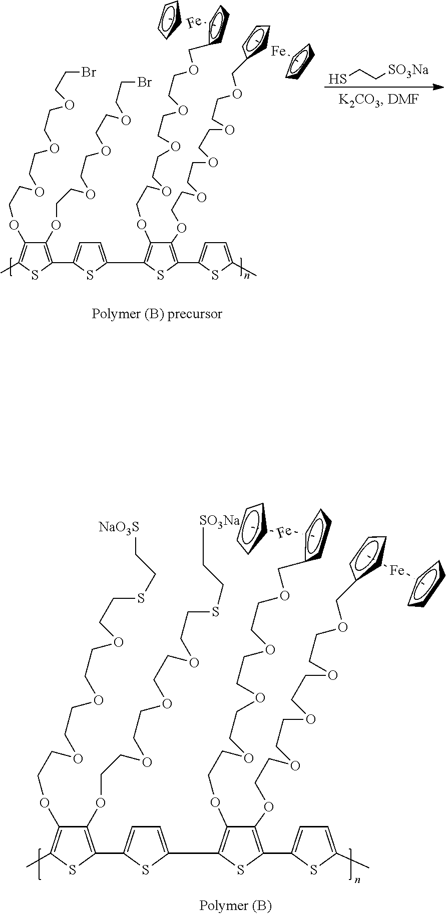

0.5 mol of monomer 3, 0.5 mol of monomer 4, 1.0 mol of 2,5-thiophene-diboronic acid, Pd(PPh₃)₄ (10% of monomer 3), and K₂CO₃ were placed in a two-necked flask under a nitrogen atmosphere. 20 ml of THF and 6 ml of water were added, and the reaction mixture was heated to 70° C. for about 20 hrs. The reaction mixture was then cooled to room temperature and poured into CH₃OH, which resulted in the formation of a precipitate. The collected precipitate was washed with CH₃OH several times and dried by vacuum to give polymer (B) precursor as a dark sticky oil. The polymer (B) precursor was then dissolved in anhydrous DMF, and 2 equivalents of K₂CO₃ and 2 equivalents of sodium 2-mercaptoethanesulfonate were added. The mixture was stirred at room temperature for about 16 hrs, and then transferred into a dialysis tube (CO 12,000) for dialysis against water. After dialysis, the solution in the dialysis tube was filtered to remove insoluble impurities and then freeze-dried to give polymer (B).

In accordance with the above-mentioned teachings, a number of different reagent layer formulations are evident. The following formulations are illustrative, nonlimiting embodiments of suitable reagent layer formulations consistent with the principles of the present invention.

Reagent Layer A: glucose dehydrogenase as enzyme, Fc-Thiophene-1 (A) as the redox polymer with ferrocene in the side-chain, carbon black as carbon nanomaterial, a sulfonated tetrafluoroethylene based fluoropolymer-copolymer (e.g., Nafion®) as binder, 3,6,9-trioxaundecanedioic acid, citric acid and polyethylene glycol diglycidyl ether as gel-forming cross-linkable small molecules.

Reagent Layer B: lactate oxidase, bovine serum albumin and horseradish peroxidase as enzymes, Fc-Thiophene-1 (A) as the redox polymer with a ferrocene side-chain, carbon black as carbon nanomaterial, sulfonated tetrafluoroethylene based fluoropolymer-copolymer (e.g., Nafion®) as binder, polyetheramine (e.g., Jeffamine®) and polyethylene glycol diglycidyl ether as gel-forming cross-linkable small molecules.

Reagent Layer C: glucose dehydrogenase as enzyme, Polyvinylferrocene (C) as the polymer with ferrocene in the side-chain, carbon black as carbon nanomaterial, a sulfonated tetrafluoroethylene based fluoropolymer-copolymer (e.g., Nafion®) as binder, 3,6,9-trioxaundecanedioic acid, citric acid and polyethylene glycol diglycidyl ether as gel-forming cross-linkable small molecules.

Reagent Layer D: lactate oxidase, bovine serum albumin and horseradish peroxidase as enzymes, polyvinylferrocene (C) as the polymer with ferrocene in side-chain, carbon black as carbon nanomaterial, a sulfonated tetrafluoroethylene based fluoropolymer-copolymer (e.g., Nafion®) as binder, polyetheramine (e.g., Jeffamine®) and polyethylene glycol diglycidyl ether as gel-forming cross-linkable small molecules.

Other Variations to the Above Embodiments: Fc-Thiophene-2 (B) instead of Fc-Thiophene-1 (A); 2,2′- and (Ethylenedioxy)-bis(ethylamine) instead of polyetheramine; dimethylFc-Thiopene (E) instead of Fc-Thiophene (A or B), polyethyleneimine instead of polyetheramine; Fc-Thiophene (A or B) and carboxymethyl cellulose instead of polyetheramine; addition of another layer on top of the enzyme-containing hydrogel layer comprising, for example, 4-styrene sulfonic acid-co-maleic acid and polyethylene glycol diglycidyl ether; and substitution of or a copolymer of ethylene and methacrylic acid (e.g., Surlyn®) for the sulfonated tetrafluoroethylene based fluoropolymer-copolymer (e.g., Nafion®). The above substitutions may be effected independently, or in any combination thereof.

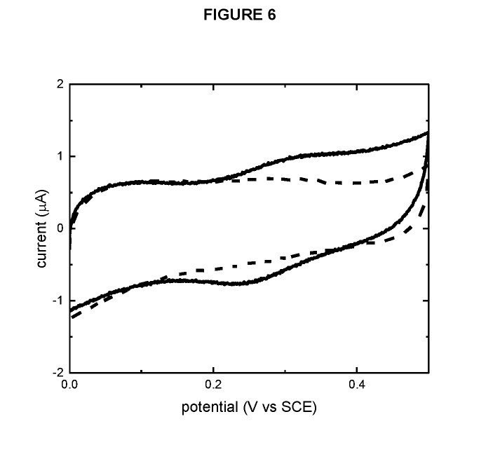

FIGS. 6-14 illustrate various characteristics and responses of glucose and lactate biosensors formulated according to the principles of the present invention, as identified in the Brief Description of the Drawings herein. By way of explanation of certain additional exemplary and nonlimiting embodiments, the following is a description of the compositions, methodology and conditions utilized in connection with the generation of the data depicted in FIGS. 6-14.

The following stock solutions were used for preparation of the sensors in the following Examples. “D.I. water” in the following description means de-ionized water with a resistance of 18 MΩ or higher.

Solution (a)—a 2:3 methanol:D.I. water solution containing: 4.0 mg/ml carbon black (VULCAN® XC72), 2.1 mg/ml Nafion®, 2.8 mg/ml 3,6,9-Trioxaundecanedioic acid, and 1.2 mg/ml sodium citrate.

Solution (b)—a 2:3 methanol:D.I. water solution containing 4.0 mg/ml carbon black (VULCAN® XC72), 2.1 mg/ml Nafion®, and 4.0 mg/ml trimethylolpropane tris[poly(propylene glycol) amine terminated] ether (Mn 440).

Solution (c)—a 2:3 methanol:D.I. water solution containing 4.0 mg/ml carbon black (VULCAN® XC72), 4.0 mg/ml polyethylenimine.

Solution (d)—12 mg/ml of Fc-thiophene-1 (A) in D.I. water

Solution (e)—50 mg/ml of poly(ethylene glycol) diglycidyl ether (Mn 500) in D.I. water.

Solution (f)—100 mg/ml of glucose dehydrogenase in pH 8.1, 10 mM HEPES buffer.

Solution (g)—a solution containing 80 mg/ml lactate oxidase and 20 mg/ml bovine serum albumin in pH 8.1, 10 mM HEPES buffer.

Solution (h)—40 mg/ml of horseradish peroxidase in pH 8.1, 10 mM HEPES buffer.

Solution (i)—10 mg/ml of poly(4-styrenesulfonic acid-co-maleic acid)sodium salt in D.I. water.

Solution (j)—50 mg/ml of ethylene glycol diglycidly ether in D.I. water.

### Example 1: (Reagent Solution for Glucose Sensor with Carbon Black)

A reagent mixture containing 100 μl of solution (a), 10 μl of solution (d), 15 μl of solution (d) and 10 μl of solution (f) was mixed thoroughly using a fine-tipped transfer pipette by applying multiple suction and release in a microvial. Once prepared, 2.5 μl of the reagent mixture was applied to a O₂ plasma treated glassy carbon electrode (diameter 3 mm) and allowed to cure for 48 h in ambient room-temperature conditions.

### Example 2: (Reagent Solution for Lactate Sensor with Carbon Black)

A reagent mixture containing 100 μl of solution (b), 10 μl of solution (d), 27 μl of solution (e), 10 μl of solution (g) and 10 μl of solution (h) were mixed thoroughly using a fine-tipped transfer pipette by applying multiple suction and release in a microvial. Once prepared, 2.5 μl of the reagent mixture was applied to a O₂ plasma treated glassy carbon electrode (diameter 3 mm) and allowed to cure for 48 h in ambient room-temperature conditions.

### Example 3: (Reagent Solution for Lactate Sensor with Carbon Black)

A reagent mixture containing 100 μl of solution (c), 10 μl of solution (d), 27 μl of solution (e), 10 μl of solution (g) and 10 μl of solution (h) were mixed thoroughly using a fine-tipped transfer pipette by applying multiple suction and release in a microvial. Once prepared, 2.5 μl of the reagent mixture was applied to a O₂ plasma treated glassy carbon electrode (diameter 3 mm) and allowed to cure for 48 h in ambient room-temperature conditions.

### Example 4: (Reagent Solution for Glucose Sensor without Carbon Black)

A reagent mixture containing 100 μl of 2.8 mg/ml 3,6,9-Trioxaundecanedioic acid and 1.2 mg/ml sodium citrate in 2:3 methanol:D.I. water, 10 μl of solution (c), 15 μl of solution (d) and 10 μl of solution (e) was mixed thoroughly using a fine-tipped transfer pipette by applying multiple suction and release in a microvial. Once prepared, 2.5 μl of the reagent mixture was applied to a O₂ plasma treated glassy carbon electrode (diameter 3 mm) and allowed to cure for 48 h in ambient room-temperature conditions.

### Example 5: (Reagent Solution for Lactate Sensor without Carbon Black)

A reagent mixture containing 100 μl of 4.0 mg/ml trimethylolpropane tris[poly(propylene glycol), amine terminated] ether (Mn 440) in 2:3 methanol:D.I. water, 10 μl of solution (c), 27 μl of solution (d), 10 μl of solution (f) and 10 μl of solution (g) were mixed thoroughly using a fine-tipped transfer pipette by applying multiple suction and release in a microvial. Once prepared, 2.5 μl of the reagent mixture was applied to a O₂ plasma treated glassy carbon electrode (diameter 3 mm) and allowed to cure for 48 h in ambient room-temperature conditions.

### Example 6: (Adding Second Layer to Example 3)

A reagent mixture containing 1 ml of solution (i) and 50 μl of solution (j) is thoroughly mixed and a 20 μl of the mixture is drop-cast onto the electrode preformed with the layers mentioned in Example 3.

### Electrochemical Experiments

The electrochemical experiments were conducted in pH 5.3, 0.1 M potassium phosphate, 0.025 M sodium chloride. Glucose solutions and lactate solutions were prepared in the same buffer for sensor studies. Glassy carbon electrode (diameter 3 mm) were modified with the reagent layers and used as working electrodes or in this case as sensor electrode. Standard calomel electrode (SCE) was used as the reference electrode and a platinum wire was used as the counter electrode.

In a typical experiment, a glassy carbon electrode modified with the reagent layer, the reference electrode and the counter electrode are immersed in an electrochemical cell filled with pH 5.3, 0.1M potassium phosphate, 0.025M sodium chloride buffer. Then the electrodes are connected to a potentiostat to control the potential and measure current. A potential of 0.4 V vs SCE was applied for glucose sensing and −0.2 V vs SCE was applied for lactate sensing. While the electrode were being applied with the specific potential, a small quantity of the analyte (glucose or lactate) stock solution is added to the buffer and mixed by turning on a magnetic stirrer for 15 s and turning off to mix the solution thoroughly. Due to the introduction of the analyte the current value changes and attains a value, which is the measure of the analyte concentration in the buffer solution.

As illustrated in FIG. 6 the response of a glucose biosensor formulated according to Example 4, without carbon black, was measured both before the introduction of glucose as an analyte to the sensor, and afterwards. More specifically, the electrode included a reagent layer with glucose dehydrogenase, and Fc-Thiophene-1 (A), without carbon black, before (solid line) and after (broken line) glucose detection for 12 min at 0.4 V, vs a reference electrode (SCE), in pH 5.3, 0.1 M potassium phosphate, 26 mM sodium chloride, 10 mM glucose solution. Before the introduction of glucose, the response is indicated by the solid line in the figure. The shape of the curve depicted therein is indicative of the presence of the redox polymer within the reagent layer of the biosensor. After the introduction of glucose, and its reaction there with, the responses then again measured as indicated by the broken line. Is apparent from FIG. 6, the response of the biosensor, including the peaks characteristic of the presence of the redox polymer, are not strongly manifested. This is believed to be indicative of the degradation or leaching of the redox polymer from the reagent layer of the biosensor.

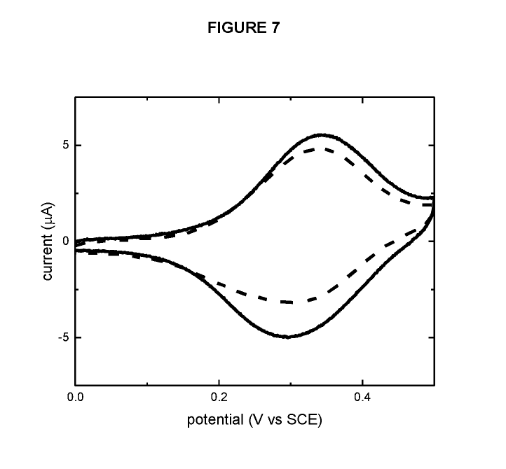

As illustrated in FIG. 7, the response of a glucose biosensor formulated according to Example 1, so as to include carbon black was also measured before and after the introduction of glucose as an analyte thereto. More specifically, the electrode a reagent layer comprising glucose dehydrogenase, Fc-Thiophene-1, and carbon black, with glucose detection for 60 min at 0.4 V, vs a reference electrode (SCE), in pH 5.3, 0.1M potassium phosphate, 26 mM sodium chloride, 10 mM glucose solution. As evident from FIG. 7, the response of the biosensor after the introduction of glucose (broken line) mimics the response of the biosensor prior to the introduction of glucose. This is believed to be indicative of the continuing presence of the redox polymer within the reagent layer of the biosensor, or in other words a lack of leaching or degradation of the redox polymer. Thus, these experiments are believed to demonstrate that carbon nanomaterial (e.g., carbon black) can impart a stabilizing effect to the redox polymer contained in the reagent layer of the biosensor.

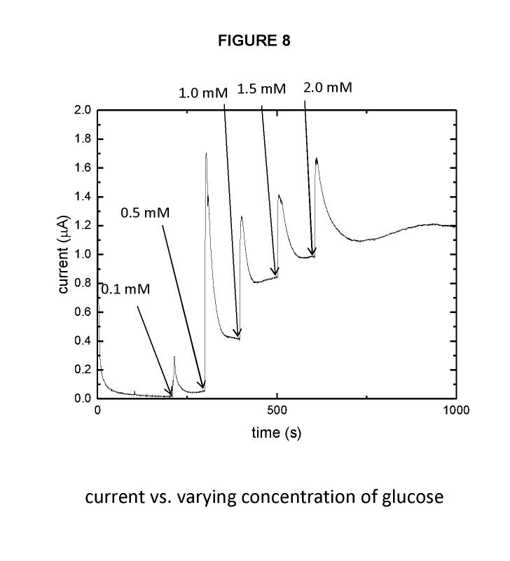

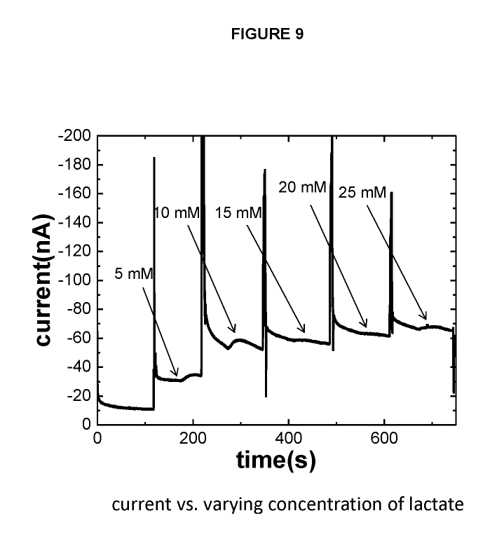

FIG. 8 illustrates the response of a glucose biosensor formulated according to Example 1 when exposed to increasing concentrations of glucose over time, as indicated therein. Likewise, FIG. 9 illustrates the response of a lactate biosensor formulated according to Example 6 when exposed to increasing concentrations of lactate over time. The data was collected under conditions specified above under the heading Electrochemical Experiments.

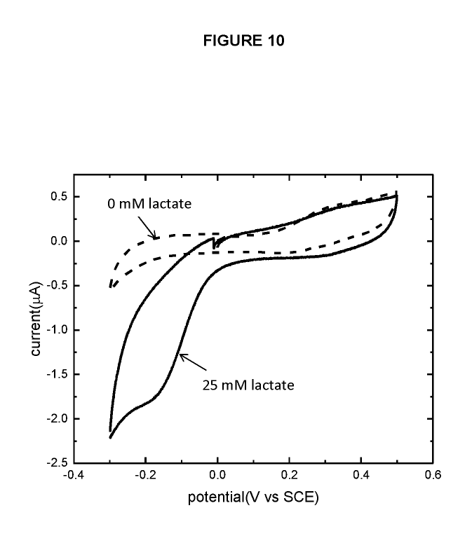

FIG. 10 illustrates the response of a lactate biosensor formulated according to Example 2 (i.e., lactate oxidase, horseradish peroxidase, Fc-Thiophene-1 (A), and carbon black) both without exposure to lactate (broken line), and with exposure to a 25 mmol lactate solution (solid line). The sensor, the electrical response of which is depicted in FIG. 10, contains carbon nanomaterial in the form of carbon black. The data was collected under conditions specified above under the heading Electrochemical Experiments.

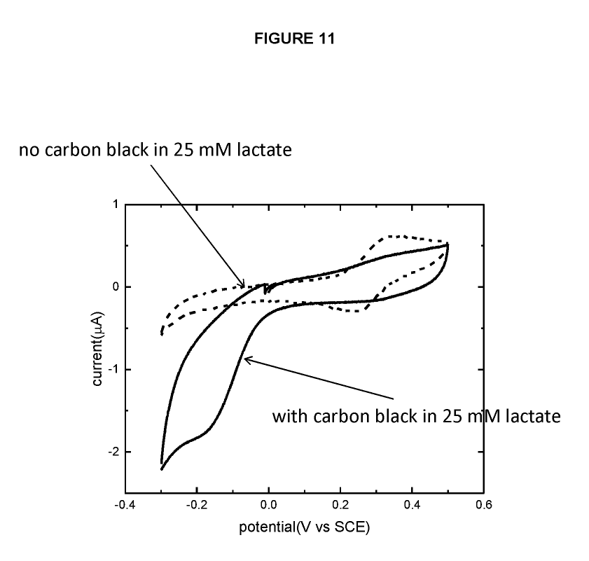

FIG. 11 depicts the response of lactate biosensor formulated according to Example 2 so as to exclude carbon black (broken line), as well as according to Example 5, including carbon black (solid line). More specifically, the a biosensor included a reagent layer comprising lactate oxidase, horseradish peroxidase, Fc-Thiophene-1 (A), with and without carbon black, Trimethylolpropane tris[poly(propylene glycol), amine terminated] ether Mn 440, poly(ethylene glycol) diglycidyl ether Mn 500, and a reference electrode, in a 25 mM lactate solution. The data was collected under conditions specified above under the heading Electrochemical Experiments.

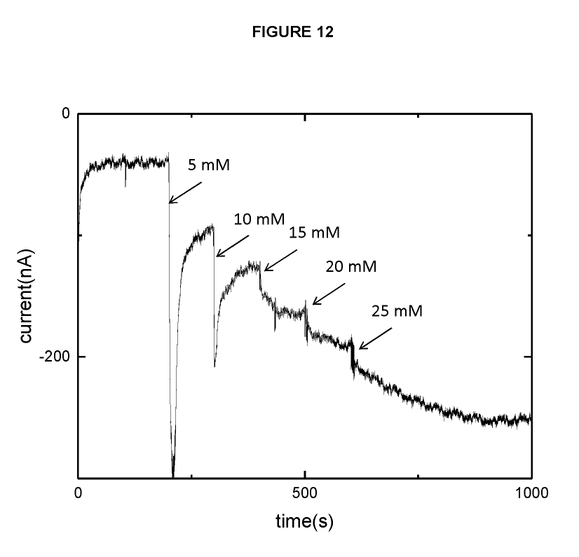

FIG. 12 depicts the current (nA) response of a lactate biosensor constructed according to Example 3 when exposed to increasing amounts or concentrations of lactate over time. The data was collected under conditions specified above under the heading Electrochemical Experiments.

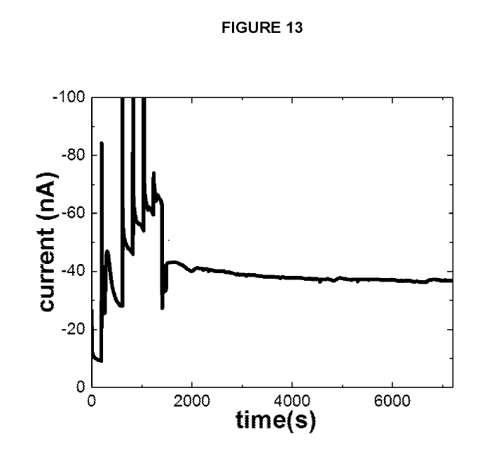

FIG. 13 depicts the current response of a lactate biosensor constructed according to the present invention over time when exposed to a sample containing a concentration of 12 mM of lactate. The current response decreased by approximately 15% over 90 minutes. The data was collected under conditions specified above under the heading Electrochemical Experiments.

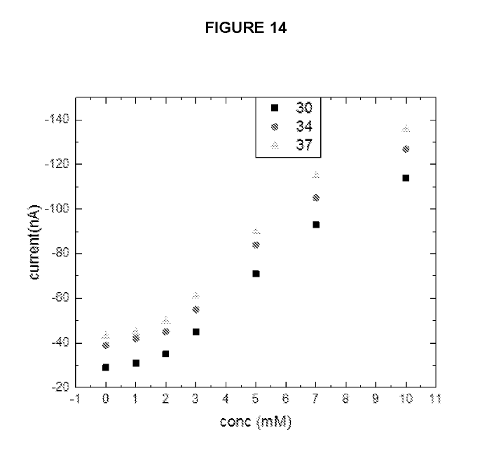

FIG. 14 depicts the current response of a lactate biosensor constructed according to the present invention when exposed to different concentrations of lactate at different temperatures (▪=30° C.; ●=34° C.; and ▴=37° C.). The data was collected under conditions specified above under the heading Electrochemical Experiments.

FIG. 9 depicts the response of lactate biosensor formulated so as to include carbon black and polyethylenimine (Example 3) and an additional layer as described in Example 6. The data shows change in current for increment of lactate concentration from 5 mM to 25 mM in a step-like fashion. FIG. 13 shows the response obtained at an electrode with the same composition exhibiting both change in current for increment of lactate concentration from 5 mM to 25 mM and long-term stability for a concentration of 12 mM lactate. This long-term stability is a crucial property for a biosensor for real-time monitoring of biochemical signals. The change in current values for various lactate concentrations at different temperature values is shown for the same composition in FIG. 14. The current values lie closely (within 20 nA variation). This is an advantageous feature making the biosensor suitable for application in environments where the temperature fluctuates between 30-37° C.

Other embodiments within the scope of the claims herein will be apparent to one skilled in the art from consideration of the specification or practice of the invention as disclosed herein. It is intended that the specification be considered exemplary only, with the scope and spirit of the invention being indicated by the claims.

As various changes could be made in the above methods and compositions without departing from the scope of the invention, it is intended that all matter contained in the above description shall be interpreted as illustrative and not in a limiting sense.

None of the features recited herein should be interpreted as invoking 35 U.S.C. § 112, ¶6, unless the term “means” is explicitly used.

1. A biosensor comprising:
at least one electrode surface;
a reagent layer disposed on the at least one electrode surface, the reagent layer comprising:
a redox enzyme,
a redox polymer, and
a first layer of gel.

2. The biosensor of claim 1, wherein the reagent layer further comprises a carbon material.

3. The biosensor of claim 2, wherein the carbon material comprises carbon black.

4. The biosensor of claim 1, wherein the redox polymer comprises:
a backbone comprising a conjugated polymer;
a first side chain attached to the backbone, the first side chain comprising a ferrocene group, a tetrathiafulvalene group or derivatives thereof;
a second side chain attached to the backbone, the second side chain comprising an organic acid or a salt of an organic acid; and
at least one of the first and second side chains comprising at least one of a carbon atom, a nitrogen atom, an oxygen atom, and a sulfur atom.

5. The biosensor of claim 4, wherein the conjugated polymer comprises at least one of a polythiophene, a polyaniline, a polyacetylene, a poly(p-phenylene), a polypyrrole and derivatives thereof.

6. The biosensor of claim 4, wherein the first chain comprises 5 to 40 atoms between the ferrocene group, the tetrathiafulvalene group, or the derivatives thereof, and the conjugated polymer.

7. The biosensor of claim 4, wherein at least one of the first and second side chains comprise an ethylene oxide group.

8. The biosensor of claim 4, wherein the second side chain comprises a carboxylic acid group, a carboxylate group, a sulfonic acid group or a sulfonate group.

9. The biosensor of claim 1, wherein the redox polymer is water soluble.

10. The biosensor of claim 1, wherein the redox enzyme comprises at least one of: a dehydrogenase, a reductase, an oxidase, an oxygenase, a peroxidase, a catalase and a transhydrogenase.

11. The biosensor of claim 1, wherein the gel comprises a hydrogel.

12. The biosensor of claim 2, wherein the first layer of gel is a hydrogel, and the biosensor further comprising a second layer of hydrogel on top of the first layer of hydrogel.

13. The biosensor of claim 12, wherein at least the second layer of hydrogel is formed from a polymer having an anionic functional group and a cross-linking agent.

14. A method of manufacturing a biosensor of claim 1, wherein the method comprises: depositing the reagent layer on the electrode surface in a single application step.

15. The method of claim 14, wherein the single application step comprises drop casting.

16. The method of claim 14, further comprising: crosslinking the gel.
